(19) United States
(12) Patent Application Publication
(10) Pub. No.: US 2022/0104716 A1
(43) Pub. Date: Apr. 7, 2022

(54) PROTECTIVE BARRIER AND METHODS FOR PROTECTING A PATIENT DURING BLOOD PRESSURE MEASUREMENT

(71) Applicant: Kenneth D. Downey, SR., Le Roy, IL (US)

(72) Inventor: Kenneth D. Downey, SR., Le Roy, IL (US)

(21) Appl. No.: 17/064,232

(22) Filed: Oct. 6, 2020

(51) Int. Cl. A61B 5/022; A61B 90/00

(52) U.S. Cl. CPC A61B 5/02233; A61B 90/08

(57) ABSTRACT

A protective barrier for protecting an arm of a patient during a blood pressure measurement is disclosed. The protective barrier includes a waterproof layer defining an exterior waterproof surface of the protective barrier and an absorbent layer defining an interior absorbent surface of the protective barrier. An adhesive layer is disposed between the waterproof layer and the absorbent layer to bond the waterproof layer and absorbent layer together to form the protective barrier. The protective barrier also includes at least one adhesive tab extending outward from an edge of the protective barrier to removably attach the adhesive tab to a portion of the protective barrier to hold the protective barrier around the arm of the patient during the blood pressure measurement.

### FIELD OF THE INVENTION

This invention relates to apparatus and methods for protecting a patient during blood pressure measurement.

### BACKGROUND OF THE INVENTION

Blood pressure measurement typically includes the placement of a blood pressure device around the patient's arm. Prior to measurement, a blood pressure cuff is placed around the patient's arm and adjusted such that the blood pressure cuff is in direct contact and tightly wrapped around one of the patient's arm. Once the blood pressure cuff is properly positioned, the blood pressure cuff is inflated such that the blood pressure cuff tightens around the patient's arm. The patient's blood pressure is then measured while the blood pressure cuff remains tightly wrapped around and in direct contact with the patient's arm. Because the blood pressure cuff remains in direct contact with the patient's arm during blood pressure measurement, it may be difficult to keep undesired material from being transferred between the patient and the blood pressure cuff.

### SUMMARY

A protective barrier for protecting an arm of a patient during a blood pressure measurement is disclosed. The protective barrier includes a waterproof layer defining an exterior waterproof surface of the protective barrier and an absorbent layer defining an interior absorbent surface of the protective barrier. The protective barrier also includes an adhesive layer disposed between the waterproof layer and the absorbent layer, wherein the adhesive layer bonds the waterproof layer and the absorbent layer together to form the protective barrier. The protective barrier also includes at least one adhesive tab extending outward from an edge of the protective barrier, wherein the at least one adhesive tab removably attaches to a portion of protective barrier to hold the protective barrier around the arm of the patient during the blood pressure measurement.

A method of protecting an arm of a patient during a blood pressure measurement is also disclosed. The method includes wrapping a protective barrier around the arm of the patient prior to taking the blood pressure measurement, overlapping a first edge with a second edge of the protective barrier to form a tubular structure around the arm of the patient, and wrapping a blood pressure device around the arm of the patient such that the blood pressure device contacts an exterior surface of the protective barrier.

A protective barrier sheet including a plurality of protective barriers for protecting an arm of a patient during a blood pressure measurement is also disclosed. The protective barrier sheet includes a first protective barrier of the plurality of protective barriers, a second protective barrier of the plurality of protective barriers adjacent to the first protective barrier, and a perforation formed between the first protective barrier and the second protective barrier. The first protective barrier is separated from the second protective barrier by tearing the protective barrier sheet along the perforation and each protective barrier of the first protective barrier and the second protective barrier includes a waterproof layer defining an exterior waterproof surface of the protective barrier and an absorbent layer defining an interior absorbent surface of the protective barrier. The protective barrier sheet also includes an adhesive layer disposed between the waterproof layer and the absorbent layer, wherein the adhesive layer bonds the waterproof layer and the absorbent layer together to form the protective barrier, and at least one adhesive tab extending outward from an edge of the protective barrier, wherein the at least one adhesive tab removably attaches to a portion of protective barrier to hold the protective barrier around the arm of the patient during the blood pressure measurement.

### BRIEF DESCRIPTION OF THE DRAWINGS

FIG. 1 is a perspective view of a standard blood pressure device.

FIG. 2A is a top view of a protective barrier adapted for use with a blood pressure device, in accordance with an exemplary embodiment of the present disclosure.

FIG. 2B is a cross-sectional view of the protective barrier of FIG. 2A, showing a plurality of layers of the protective barrier, in accordance with an exemplary embodiment of the present disclosure.

FIG. 3 is a perspective view of a plurality of protective barriers formed in a continuous sheet, and showing the protective barriers separated by a perforation, in accordance with an exemplary embodiment of the present disclosure.

FIG. 4 is a perspective view of an apparatus for manufacture of the protective barrier of FIGS. 2A, and 3, in accordance with an exemplary embodiment of the present disclosure.

FIG. 5 is a perspective view of utilizing the protective barrier of FIG. 2A during the blood pressure measurement of a patient, in accordance with an exemplary embodiment of the present disclosure.

### DETAILED DESCRIPTION OF THE INVENTION

This invention is based, at least in part, on a flexible protective barrier for use with a blood pressure device. The flexible protective barrier provides an intermediate layer between the patient and blood pressure device that reduces or eliminates the transfer of undesired materials between the patient and the blood pressure device.

### I. BLOOD PRESSURE DEVICE

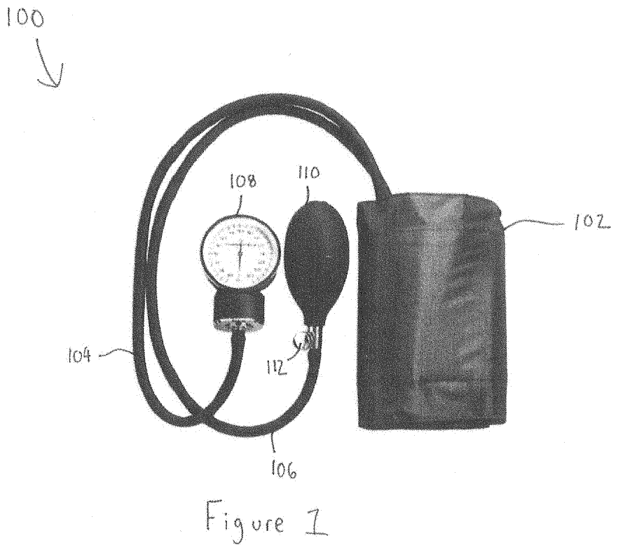

FIG. 1 is a perspective view of a blood pressure device 100 utilized to measure and/or monitor the blood pressure of a patient or other such user. The blood pressure device 100 comprises a blood pressure cuff 102, a first flexible tubing 104 connected at one end of the first flexible tubing 104 to the blood pressure cuff 102, and a second flexible tubing 106 fluidically connected at one end of the second flexible tubing 106 to the blood pressure cuff 102. A pressure gauge 108 fluidically connected to a second end of the first flexible tubing 104. An air pump 110 fluidically connected to the second end of the second flexible tubing 106. The air pump 110 can be a squeezable bulb or other such air pressure generating device that is utilized to inflate an inflatable chamber (not shown) of the blood pressure cuff 102. The pressure gauge 108 measures a pressure inside of the inflatable chamber of the blood pressure cuff 102. The blood pressure device 100 further comprises a pressure release valve 112 fluidically connected to the second flexible tubing 106 such that opening the pressure release valve 112 causes the inflatable pressure inside of the blood pressure cuff 102 to deflate. The blood pressure device 100 is shown and described as an example, it will be appreciated that the present apparatus can also be used with other types of devices.

### II. PROTECTIVE BARRIER

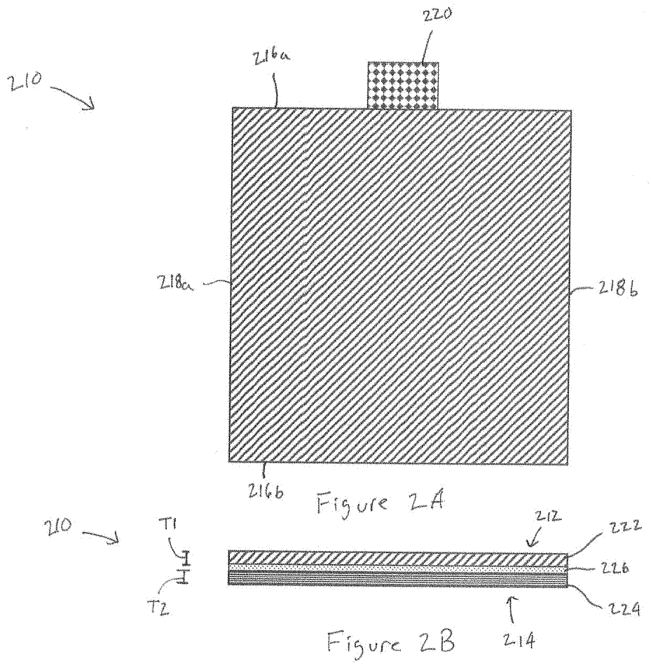

FIGS. 2A, and 2B show an embodiment of a protective barrier 210 for use with a blood pressure measurement device, such as the blood pressure device 100 of FIG. 1. In various embodiments, the protective barrier 210 is utilized during blood pressure measurement to provide an intermediate layer or separation layer between the patient's arm and the blood pressure device 100. For example, the protective barrier 210 is wrapped around the patient's arm before the placement of the blood pressure device 100 onto the patient's arm. As such, the protective barrier 210 provides an intermediate layer between the patient's arm and the blood pressure cuff 102 of the blood pressure device 100. In various embodiments, the protective barrier 210 reduces or eliminates the transfer of any fluid, bacteria, dust, dirt, or other undesirable material between the patient's arm and the blood pressure cuff 102 because the blood pressure device 100 does not directly contact the patient's arm. In various embodiments, the protective barrier 210 may also reduce or eliminate the transfer of any fluid, bacteria, dust dirt, or other undesirable material between the patient's clothing and the blood pressure cuff 102 or other portion of the blood pressure device 100.

For example, the protective barrier 210 prevents sweat, blood, bacteria, dirt, dust, dry skin, or other such material present on the blood pressure cuff 102 from being transferred from the blood pressure device 100 to the patient's arm and/or clothing. The protective barrier 210 also prevents sweat, blood, bacteria, dirt, dust, dry skin, or other such material present on the patient's arm and/or clothing from being transferred from the patient to the blood pressure device 100. In various embodiments, the protective barrier 210 reduces the amount of time spent cleaning blood pressure devices between each use. Additionally, the protective barrier 210 can be utilized as a personal protective equipment device utilized to minimize the exposure and/or transfer of hazardous and/or infectious material between a patient and a health care worker. As such, the protective barrier 210 may improve the patient experience (i.e., relax or reduce anxiety) by providing a visible protective barrier that prevents transfer of hazardous and/or infectious contaminants between the blood pressure cuff and the patient. In various embodiments, the protective barrier 210 may also improve the comfort of the patient during blood pressure measurement by providing a more comfortable interface between the patient and the blood pressure device 100. The protective barrier 210 acts as an intermediate layer between the patient's arm and the blood pressure device 100 and eliminates direct contact of the blood pressure device 100 and the surface of the patient's arm.

In various embodiments, the protective barrier 210, forms a flexible sheet or panel including a first surface 212 and a second surface 214 opposite the first surface 212. In various embodiments, the protective barrier 210 is substantially rectangular in shape, however it will be appreciated that other shapes of the protective barrier are possible. In the illustrated example embodiment, the rectangular shape of the protective barrier 200 is defined by a first edge 216a substantially parallel to and spaced apart from a second edge 216b, and a third edge 218a substantially parallel to and spaced apart from a fourth edge 218b. The third edge 218a connects a first end of the first edge 216a to a first end of the second edge 216b. The fourth edge 218b connects a second end of the first edge 216a and a second end of the second edge 216b.

In various embodiments, the protective barrier 210 includes dimensions that enable the flexible panel to wrap around the patient's arm and extend axially outward from the blood pressure device 100. For example, the first and second edges 216a and 216b extending between the third and fourth edges 218a and 218b define a width of the protective barrier 210 that is larger than a width of the blood pressure device 100. In some embodiments, dimensions of the protective barrier 210 are defined to produce a protective barrier width of at least 4 to 12 inches. As such the width defined by the first and second edges 216a and 216b extending between the third and fourth edges 218a and 218b can be sized such that the protective barrier 210 extends axially outward from each side of the blood pressure device 100 a desired amount (e.g., 1 inch, 2 inches, etc.). That is, the protective barrier 210 can be sized to have a width larger than the width of the blood pressure device 100 such that when the protective barrier 210 is wrapped around the patient's arm the barrier extends at least 1 inch beyond each edge of the blood pressure cuff 102 of the blood pressure device 100. It will be understood that the protective barrier can be alternatively sized to extend a more or less than 1 inch from each edge of the blood pressure cuff of the blood pressure device. For example, the protective barrier may be sized such that the barrier extends 2 or more inches from each edge of the blood pressure cuff.

In various embodiments, the protective barrier 210 also includes dimensions that enable the flexible panel to wrap completely around a circumference of the patient's arm. For example, the third and fourth edges 218a and 218b define a length of the protective barrier 210 that is larger than the circumference of the patient's arm. In some embodiments, the length of the protective barrier 210 defined by the third and fourth edges 218a and 218b can be at least 8 to 16 inches. As such, the length of the third and fourth edges 218a and 218b can be sized such that the first and second edges 216a and 216b may overlap one another such that the protective barrier 210 wraps completely around the circumference of the patient's arm.

In various embodiments, one or more adhesive tabs 220 outwardly extend from an edge of the protective barrier 210. More specifically, the one or more adhesive tabs 220 extend outward from at least one of the first edge 216a of the protective barrier 210. It should be appreciated that in other embodiments, the one or more adhesive tabs can additionally or alternatively extend outward from the second edge 216b, the third edge 218a, the fourth edge 218b, the first surface 212, the second surface 214, or other such portion of the protective barrier 210. In various embodiments, the one or more adhesive tabs 220 have a removable backing (not shown) that covers an adhesive layer (not shown) of the adhesive tab 220 when not in use. As such, the removable backing can be removed from the adhesive tab 220 to stick or otherwise attach the first edge 216a to another portion of the protective barrier 210. For example, the protective barrier 210 may be wrapped around a patient's arm such that the first edge 216a and the second edge 216b are positioned adjacent and/or overlapping one another. As such, the one or more adhesive tabs 220 can be utilized to removably attach the first edge 216a to an area along and/or interior to the second edge 216b of the protective barrier 210. In other words, the protective barrier 210 can be wrapped around the patient's arm forming a tubular structure that at least a portion of the patient's arm extends through. In various embodiments, the adhesive tab 220 is utilized to hold the protective barrier 210 around the patient's arm during measurement of the patient's blood pressure. After the blood pressure measurement is complete, the adhesive tab 220 can be unattached to remove the protective barrier 210 from the patient's arm. In various embodiments, the protective barrier 210 is a single use device such that after use, the protective barrier 210 is properly disposed of with other suitable medical waste.

In various embodiments, the protective barrier 210 is formed by a plurality of layers built-up or stacked on top of one another to form a multi-layer sheet or panel. More specifically, FIG. 2B shows a cross-sectional view of the protective barrier 210 including a first layer 222 having a thickness T1, a second layer 224 having a thickness T2, and a third layer 226 having a thickness T3 (not labeled) disposed between the first and second layers 222 and 224. In various embodiments, the first layer 222 comprises a flexible waterproof material formed from one or more plastic materials such as polyethylene, polypropylene, polyethylene terephthalate, or any other such waterproof material or combination thereof. As such, the first layer 222 forms a waterproof layer of the protective barrier 210 that prevents liquid and other such material from penetrating or otherwise being transported through the first layer 222 of the protective barrier 210. In various embodiments, the first surface 212 of the protective barrier 210 defines an outer waterproof surface of the protective barrier 210. In various embodiments, the second layer 224 comprises a flexible absorbent layer formed from one or more of a paper material, natural fabric material, synthetic fabric material, or any other such flexible absorbent material or combination thereof that absorbs or collects any liquid (e.g., sweat, water, etc.) present on the surface of the patient's arm. In various embodiments, the second surface 214 of the protective barrier 210 defines an interior absorbent surface of the protective barrier 210.

In various embodiments, the third layer 226 comprises an adhesive material applied to at least one of the first layer 222 and the second layer 224 such that the first layer 222 and the second layer 224 are fixedly attached to one another. For example, the third layer 226 comprises a medical grade adhesive that bonds the first layer 222 (e.g., the waterproof layer) to the second layer 224 (e.g., the absorbent layer) of the protective barrier 210. In various embodiments, the adhesive material of the third layer 226 is sprayed or otherwise dispensed onto at least one of the first and second layers 222 and 224. In various embodiments, upon curing or drying, the adhesive material forms a continuous layer that bonds the first layer 222 and the second layers 224 together.

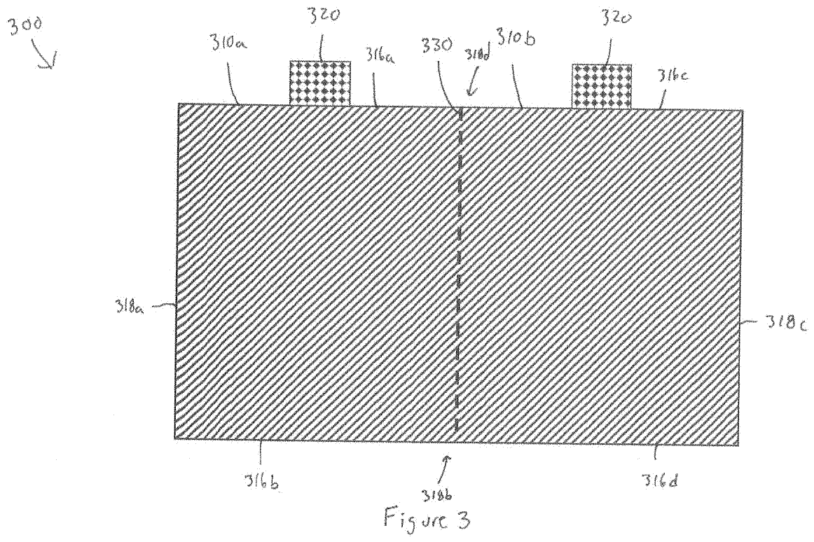

Referring now to FIG. 3, in certain embodiments, the protective barrier can formed in a continuous sheet including a plurality of protective barrier panels that can be separated from one another to form individual protective barriers (e.g., protective barrier 210 of FIGS. 2A and 2B). More specifically FIG. 3 shows a protective barrier sheet 300 including a first protective barrier 310a, a second protective barrier 310b, and a perforation 330 formed through at least a portion of the thickness of the protective barrier sheet 300. As such, the first protective barrier 310a can be separated from the second protective barrier 310b along the perforation 330 to form separate protective barriers similar to the protective barrier 210 of FIGS. 2A and 2B.

In various embodiments, the first protective barrier 310a and the second protective barrier 310b are substantially rectangular in shape and when separated along the perforation 330, the panels are substantially the same size (e.g., have same length and width). In certain other embodiments, the perforation may be formed in the protective barrier such that when separated, the panels are different sizes (e.g., have different length and/or width).

In various embodiments, the first protective barrier 310a is defined by a first edge 316a substantially parallel to and spaced apart from a second edge 316b, and a third edge 318a substantially parallel to and spaced apart from a fourth edge 318b. The third edge 318a connects a first end of the first edge 316a to a first end of the second edge 316b. The fourth edge 318b connects a second end of the first edge 316a and a second end of the second edge 316b. In various embodiments, the second protective barrier 310b is defined by a fifth edge 316c substantially parallel to and spaced apart from a sixth edge 316d, and a seventh edge 318c substantially parallel to and spaced apart from an eighth edge 318d. The seventh edge 318c connects a first end of the fifth edge 316c to a first end of the sixth edge 316d. The eighth edge 318d connects a second end of the fifth edge 316c and a second end of the sixth edge 316d. In such embodiments, prior to separation of the first and second protective barriers 310a and 310b along the perforation 330, the first edge 316a and the fifth edge 316c, form a first continuous edge along the length of the protective barrier sheet 300. Similarly, prior to separation of the first and second protective barriers 310a and 310b along the perforation 330, the second edge 316b and the sixth edge 316d form a second continuous edge along the length of the protective barrier sheet 300, the second continuous edge is substantially parallel to and spaced apart from the first continuous edge.

In various embodiments, the protective barriers 310a and 310b include dimensions that enable the barriers to wrap around the patient's arm and extend axially outward from the blood pressure device. For example, the first and second edges 316a and 316b extending between the third and fourth edges 318a and 318b of protective barrier 310a and the fifth and sixth edges 316c and 316d extending between the seventh and eighth edges 318 c and 318d of protective barrier 310b define a width of the protective barriers that is larger than a width of the blood pressure device. In some embodiments, the width of the protective barrier defined by the first and second edges 316a and 316b and the fifth and sixth edges 316c and 316d is at least 4 to 10 inches. As such the width defined by the first and second edges 316a and 316b and the fifth and sixth edges 316c and 316d can be sized such that the protective barriers 310a and 310b extend axially outward from both sides of the blood pressure device a desired amount (e.g., 1 inch, 2 inches, etc.). As such, the protective barriers 310a and 310b can be sized to have a width larger than the width of the blood pressure device such that the protective barriers 310a and 310b extend beyond the blood pressure cuff of the blood pressure device.

In various embodiments, the protective barriers 310a and 310b also include dimensions that enable the flexible panel to wrap completely around a circumference of the patient's arm. For example, the third and fourth edges 318a and 318b of protective barrier the 310a and seventh and eighth edges 318c and 318d of the protective barrier 310b define a length of the protective barriers that is larger than the circumference of the patient's arm. In some embodiments, the length of the protective barriers 310a and 310b defined by the third and fourth edges 318a and 318b and the seventh and eighth edges 318c and 318d can be at least 8 to 16 inches. As such, the protective barriers 310a and 310b can be sized to have a length that enables the protective barriers 310a and 310b to completely wrap around the circumference of the patient's arm.

In various embodiments, one or more adhesive tabs 320 outwardly extend from an edge of the protective barriers 310a and 310b. In the illustrated example embodiment, the one or more adhesive tabs 320 extend outward from the first edge 316a of the protective barrier 310a and the fifth edge 316c of the protective barrier 310b. It should be appreciated that in other embodiments, the one or more adhesive tabs 320 can additionally or alternatively extend outward from the second edge 316b, the third edge 318a, the fourth edge, 318b, the sixth edge 316d, the seventh edge 318c, the eighth edge 318d, or other such portion of the protective barriers 310a and 310b. In various embodiments, the one or more adhesive tabs 320 have a removable backing (not shown) that covers an adhesive layer (not shown) of the adhesive tab when not in use. As such, the removable backing can be removed from the adhesive tabs 320 to stick or otherwise attach the first edge 316a and the fifth edge 316c to another portion of the protective barriers 310a and 310b, respectively.

For example, the first protective barrier 310a may be wrapped around a patient's arm such that the first edge 316a and the second edge 316b are positioned adjacent and/or overlapping one another. As such, the one or more adhesive tabs 320 can be utilized to attach the first edge 316a to an area along and/or interior to the second edge 316b of the first protective barrier 310a. The second protective barrier 310b may be wrapped around a different patient's arm such that the fifth edge 316c and sixth edge 316d are positioned adjacent and/or overlapping one another. As such, the one or more adhesive tabs 320 can be utilized to attach the fifth edge 316c to an area along and/or interior to the sixth edge 316d of the second protective barrier 310b. In other words, the first and second protective barriers 310a and 310b can be wrapped around a patient's arm forming a tubular structure that at least a portion of the patient's arm extends through. The adhesive tab is utilized to hold the first and second protective barriers 310a and 310b around the patient's arm during measurement of the patient's blood pressure.

### III. SYSTEM FOR MANUFACTURING A PROTECTIVE BARRIER

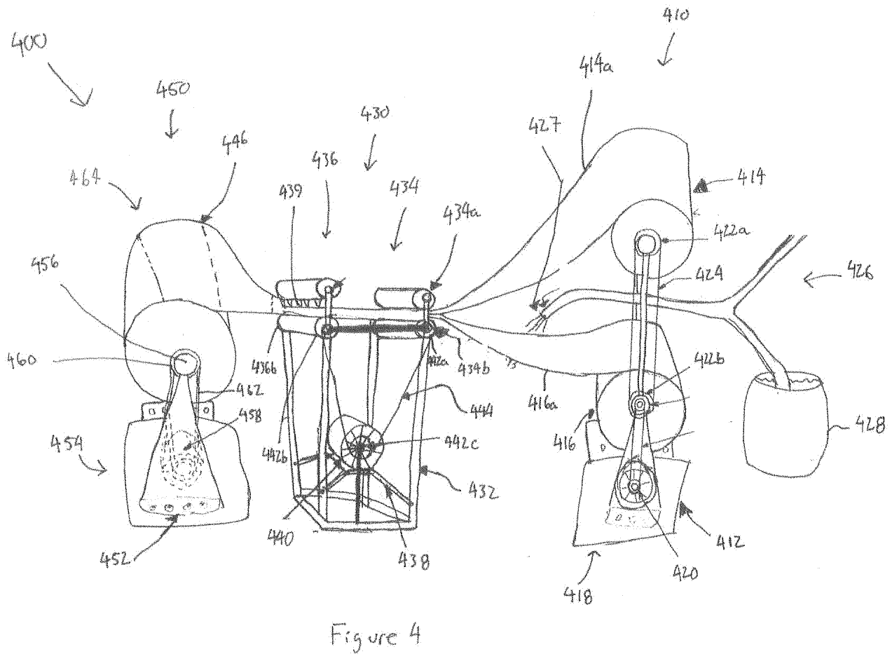

FIG. 4 is a perspective view of a manufacturing system 400 for the automated bulk manufacture of a protective barrier, such as the protective barrier 210 and 310 of FIGS. 2A, 2B. and 3. In various embodiments, the manufacturing system 400 includes a raw materials device 410, a material bonding device 430 aligned with and spaced apart from the raw materials device 410, and a finished material collection device 450 aligned with and spaced apart from the material bonding device 430. In the illustrated example embodiment, the manufacturing system 400 receives a first layer of material, such as a plastic film material and a second layer of material, such as an absorbent material that are aligned on top of each other and fed into the material bonding device 430 to form the protective barrier. Once bonded together by the material bonding device 430, the protective barrier is received and collected by the finished material collection device 450. In certain embodiments, the manufacturing system 400 forms the protective barrier in a continuous sheet that is collected and rolled into bulk rolls of the protective barrier by the finished material collection device 450. It should be appreciated that the manufacturing system 400 is not shown to scale.

In various embodiments, the raw materials device 410 includes a material stand 412 configured to hold a first material layer roll 414 (e.g., waterproof layer) and a second material layer roll 416 (e.g., absorbent layer). The material stand 412 further includes a drive system 418 connected to and configured to rotate the first and second material layer rolls 414 and 416. For example, the drive system 418 includes a drive motor 420 and one or more pulleys 422 operatively connected to the drive motor 420 by a belt 424. For example, the one or more pulleys 422 includes a first pulley 422a operatively connected to the first material layer roll 414, a second pulley 422b operatively connected to the second material layer roll 416. The belt 424 operatively connects the first and second pulleys 422a and 422b to the drive motor 420 such that the drive system 418 causes rotation of the first and second material layer rolls 414 and 416. In various embodiments, the drive system 418 rotates the first and second material layer rolls 414 and 416 at substantially the same speed to feed a material layer from each of the first and second material layer rolls 414 and 416 into the material bonding device 430.

In various embodiments, the raw materials device 410 also includes an adhesive dispensing system 426 configured to dispense an adhesive material layer onto at least one of a first material layer 414a and a second material layer 416a of the first and second material layer rolls 414 and 416, respectively. More specifically, the adhesive dispensing system 426 includes a dispense nozzle 427 fluidically coupled to an adhesive reservoir 428 that stores a supply of adhesive material for the manufacture of the protective barrier. In various embodiments, the dispense nozzle 427 is positioned adjacent to the first and second material layer rolls 414 and 416 such that the adhesive material layer (e.g., medical grade glue, or medical grade epoxy) can be dispensed onto a surface of at least one of the first and second material layers 414a and 416a. In various embodiments, the adhesive dispensing system 426 is connected to a compressed air system (not shown) or other such pressure source that dispenses the adhesive material layer.

For example, the material stand 412 may mount the first and second material layer rolls 414 and 416 with a substantially vertical orientation with respect to one another. That is, the material stand 412 positions the first material layer roll 414 of material above and in axial alignment to the second material layer roll 416. As such, the dispense nozzle 427 is positioned between the first and second material layer rolls 414 and 416 and dispenses the adhesive material layer onto a surface of at least one of the first and second material layers 414a and 414b. The raw materials device 410 positions the first material layer 414a on top of the second material layer 414b as the manufacturing system 400 feeds the first and second material layers 414a and 414b into the material bonding device 430. It should be appreciated that while the first material layer roll 414 is described as being mounted above the second material layer roll 416, the first and second material layer rolls may be mounted in any other such arrangement.

In various embodiments, the material bonding device 430 includes a bonding device stand 432, a set of bonding rollers 434 operatively connected to and supported by the bonding device stand 432, a set of perforation rollers 436 operatively connected to and supported by the bonding device stand 432, and a bonding drive system 438 supported by the bonding device stand 432 and operatively connected to the set of bonding rollers 434 and the set of perforation rollers 436. In the illustrated example, the set of bonding rollers 434 include a first bonding roller 434a and a second bonding roller 434b. More specifically, the first bonding roller 434a is positioned above and in axial alignment to the second bonding roller 434b. In certain embodiments, the first and second bonding rollers 434a and 434b may be vertically adjustable with respect to one another to define a gap between the first and second bonding rollers 434a and 434b. In such embodiments, the first and second bonding rollers 434a and 434b can be adjusted to define a gap dimension (e.g., vertical distance between first and second bonding rollers) that is substantially equal to or smaller than the combined thickness of the first and second material layers 414a and 416a. That is, the first and second bonding rollers 434a and 434b can be adjusted to exert a desired bonding force on the first and second material layers 414a and 416a as the material layers pass through the first and second bonding rollers 434a and 434b. Such bonding force causes the adhesive material layer to bond or otherwise attach the first and second material layers 414a and 416a together to form the protective barrier.

In various embodiments, the set of perforation rollers 436 includes a first perforation roller 436a and a second perforation roller 436b. At least one of the first and second perforation rollers 436a and 436b includes a set of protrusions 439 radially extending from the roller surface. As such, the set of protrusions is configured to imprint or otherwise form a perforation 446 in the protective barrier formed from the first and second material layers 414a and 416a. In various embodiments, a specific diameter or other such dimension may be utilized to define a circumference of the first and second perforation rollers 436a and 436b. More specifically, the first and/or second perforation roller can be configured with a circumference of 4 to 10 inches or other such circumference to define a spacing between the perforation 446 imprinted into the protective barrier. For example, a perforation roller with a 10 inch circumference will imprint the perforation 446 into the protective barrier every 10 inches to define a plurality of protective barrier panels that can be separated from one another along each of the perforations 446. While the perforation roller is described as having a 10 inch circumference, it will be understood that a different roller circumference can be used to space the perforations to form a desired panel size of the protective barrier.

In various embodiments, the bonding drive system 438 includes a drive motor 440 and one or more pulleys 442 operatively connected to the drive motor 440 by a belt 444. In the illustrated example, the one or more pulleys includes a first pulley 442a operatively connected to the second bonding roller 434b, a second pulley 442b operatively connected to the second perforation roller 436b, and a third pulley 442c operatively connected to the drive motor 440. The belt 444 connects the first and second pulleys 442a and 442b to the drive motor 440. As such, the drive motor 440 causes rotation of the first, second, and third pulleys 442a, 442b, and 442c which in turn causes rotation of the second bonding roller 434b and the second perforation roller 436b. to feed the protective barrier through the material bonding device 430 and into the finished material collection device 450.

In various embodiments, the finished material collection device 450 includes a collection stand 452, a drive system 454 supported by the collection stand 452, and a collection dowel 456 supported by the collection stand. 452. In the illustrated example, the drive system 454 includes a drive motor 458, and at least one pulley 460 operatively connected to the drive motor 458 by a belt 462. The collection dowel 456 is configured to receive the finished protective barrier from the material bonding device 430 and roll the material into a bulk protective barrier roll 464. More specifically, the collection dowel 456 is operatively connected to the pulley 460 such that the drive motor 458 causes rotation of the collection dowel 456 to roll the finished protective barrier into the bulk protective barrier roll 464 as the finished protective barrier is received from the material bonding device 430.

### IV. EXAMPLE

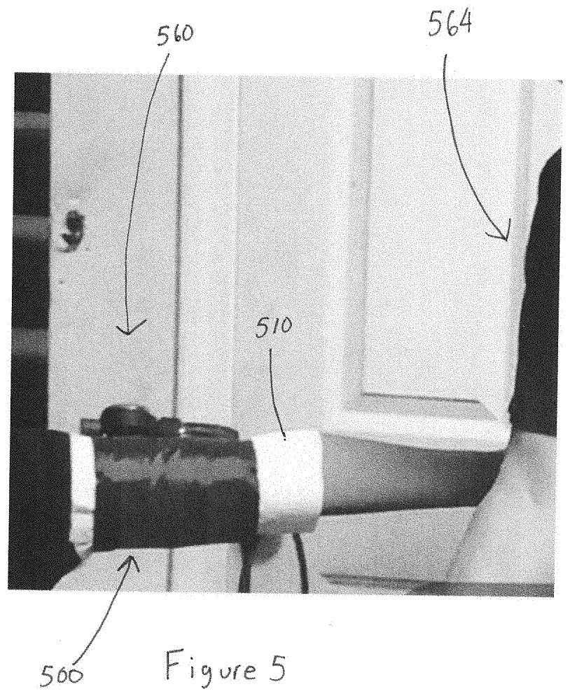

FIG. 5 illustrates use of a protective barrier 510 during measurement and/or monitoring the blood pressure of a patient 560. It should be appreciated that the protective barrier 510 is similar to the protective barrier 210 and 310 discussed above and shown in FIGS. 2A, 2B, and 3. In various embodiments, the protective barrier 510 is wrapped around the patient's arm to isolate or protect the patient 560 during blood pressure measurement and/or monitoring. More specifically, the protective barrier 510 is formed from multiple material layers (e.g., first layer 222, second layer 224, and third layer 226 of FIG. 2B) to reduce and/or eliminate the transfer of fluids, bacteria, dust, dirt, and other undesired material between the blood pressure device 500 and the arm of the patient 560. For example, the protective barrier 510 includes a waterproof layer (e.g., first layer 222) bonded by an adhesive layer (e.g., third layer 226) to an absorbent layer (e.g., second layer 224).

In various embodiments, the waterproof layer can be formed from a flexible plastic material or other such waterproof layer. As such, the waterproof layer prevents substantially all fluid and/or other undesirable material from being transported through the protective barrier 510. In various embodiments, the waterproof layer is partially or fully water repellant. In various embodiments, the waterproof layer is a hydrophobic layer. As such, the waterproof layer can repel liquid (e.g., sweat, water, etc.) or other such undesired material present during the blood pressure measurement. In various embodiments, the absorbent layer is formed from a paper material, a natural fabric material or synthetic fabric material, or other such material. In various embodiments, the absorbent layer is a hygroscopic layer. As such, the absorbent layer can capture liquid (e.g., sweat, water, etc.) or other such material present on the surface of the patient's arm and hold the captured liquid within the absorbent layer. However, any liquid absorbed by the absorbent layer is stopped by the waterproof layer from penetrating or otherwise being transported through the protective barrier 510 to the outer surface of the protective barrier 510. The absorbent layer also provides a comfortable interface between the patient's arm and the protective barrier 510 that helps keep the patient's arm cool and dry during blood pressure measurement. The protective barrier 510 also prevents or minimizes rubbing or other such discomfort caused by the blood pressure device 500 when wrapped around the patient's arm.

In various embodiments, prior to blood pressure measurement, a health care professional 564 or other such individual wraps the protective barrier 510 around the arm of the patient 560. As discussed above, the protective barrier 510 includes an absorbent surface formed by the absorbent layer and a waterproof surface defined by the waterproof layer of the protective barrier 510. The health care professional 564 wraps the protective barrier 510 around the patient's arm such that the absorbent surface of the absorbent layer is in direct contact with the patient's arm. The waterproof surface of the waterproof layer is spaced above or away from the surface of the patient's arm. That is, the absorbent surface defines an interior surface of the protective barrier 510 and the waterproof surface defines an exterior surface of the protective barrier 510.

In various embodiments, the protective barrier 510 also includes one or more adhesive tabs (e.g., adhesive tabs 220 and 320 of FIGS. 2A, 2B and 3) that can be utilized to hold the protective barrier 510 in place on and/or around the patient's arm. For example, once the protective barrier 510 is positioned on patient's arm, the health care professional 564 utilizes the adhesive tabs to temporarily hold the protective barrier 510 in the wrapped position around the circumference of the patient's arm. As such, the tabs securely hold the protective barrier 510 in place during blood pressure measurement.

Once the protective barrier 510 is adjusted and suitably positioned around the patient's arm, the health care professional 564 aligns the blood pressure device 500 with the protective barrier 510 and wraps the blood pressure device 500 around the protective barrier 510 and the patient's arm. More specifically, the blood pressure device 500 is positioned such that the interior surface of the blood pressure cuff directly contacts the waterproof surface of the protective barrier 510. Thus, the protective barrier 510 creates a waterproof barrier between the blood pressure device 500 and the arm of the patient 560. In various embodiments, the waterproof layer of the protective barrier 510 reduces and/or eliminates the transfer of fluids, bacteria, and other undesirable material between the blood pressure device 500 and the patient 560. As such, the protective barrier 510 provides a more sanitary blood pressure measurement because the protective barrier eliminates the transfer of any undesired material between the patient 560 and the blood pressure device 500.

As discussed above, the protective barrier 510 can be configured with dimensions such that when positioned on the patient's arm, at least 1 inch of the protective barrier 510 extends axially outward from each side of the blood pressure device 514. That is, when the blood pressure device 500 is positioned around the patient's arm and the protective barrier 510, at least 1 inch of the protective barrier 510 extends outward from each side of the blood pressure device 500. Thus, the protective barrier 510 is configured with a width that is larger than the width of the blood pressure device 514 such that the protective barrier 510 extends beyond the outer edges of the blood pressure device 514.

Once the blood pressure measurement is complete, the health care professional 564 removes the blood pressure device 500 and the protective barrier 510 from the arm of the patient 560. In various embodiments, the protective barrier 510 is intended as a single use device and following removal from the arm of the patient 512, the protective barrier 510 is properly disposed with other suitable medically-generated waste.

From the foregoing, it should be appreciated that the above disclosed apparatus and methods may provide a disposable protective barrier that are highly effective in reducing the transfer of any undesired materials between the patient and the blood pressure device. Further, while the blood pressure device may be cleaned before and/or after each use, the protective barrier improves the sanitary conditions of the blood pressure device by reducing and/or eliminating the transfer of any undesirable materials between the patient and the blood pressure device.

It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

The terms “a”, “an” and “the” include both singular and plural referents, unless the context clearly dictates otherwise. Thus, for example, “a device” includes one device and plural devices. Unless otherwise indicated, the terms “first”, “second”, “third”, and other ordinal numbers are used herein to distinguish different elements of the present devices and methods, and are not intended to supply a numerical limit. Reference to first and second layers should not be interpreted to mean that the device only has two layers. A device having first and second elements can also include a third, a fourth, a fifth, and so on, unless otherwise indicated.

As used in the specification and appended claims, and in addition to their ordinary meanings, the terms “substantial” or “substantially” mean to within acceptable limits or degree. For example, “substantially parallel” means that one skilled in the art would consider the non-parallel orientation to be undetectable or negligible.

As used in the specification and the appended claims and in addition to its ordinary meaning, the term “approximately” means to within an acceptable limit or amount to one having ordinary skill in the art. For example, “approximately the same” means that one of ordinary skill in the art would consider the items being compared to be the same.

In the detailed description herein, for purposes of explanation and not limitation, representative embodiments disclosing specific details are set forth in order to provide a thorough understanding of the present teachings. Descriptions of known systems, devices, materials, methods of operation and methods of manufacture may be omitted so as to avoid obscuring the description of the example embodiments. Nonetheless, systems, devices, materials, and methods that are within the purview of one of ordinary skill in the art may be used in accordance with the representative embodiments.

Generally, it is understood that the drawings and the various elements depicted therein are not drawn to scale. Further, relative terms, such as “above,” “below,” “top,” “bottom,” “upper,” “lower,” “left,” “right,” “vertical” and “horizontal,” are used to describe the various elements' relationships to one another, as illustrated in the accompanying drawings. It is understood that these relative terms are intended to encompass different orientations of the protective barrier devices and/or elements in addition to the orientation depicted in the drawings. For example, if the protective barrier devices were inverted with respect to the view in the drawings, an element described as “above” another element, for example, would now be “below” that element. Likewise, if the device were rotated 90 degrees with respect to the view in the drawings, an element described as “vertical,” for example, would now be “horizontal.”

The foregoing description of exemplary or preferred embodiments should be taken as illustrating, rather than as limiting the present invention as defined by the embodiments. As will be readily appreciated, numerous variations and combinations of the features set forth above can be utilized without departing from the present invention as set forth in the embodiments. Such variations are not regarded as a departure from the scope of the invention, and all such variations are intended to be included within the scope of the following embodiments.

We claim:

1. A protective barrier for protecting an arm of a patient during a blood pressure measurement, the protective barrier comprising:
a waterproof layer defining an exterior waterproof surface of the protective barrier;
an absorbent layer defining an interior absorbent surface of the protective barrier;
an adhesive layer disposed between the waterproof layer and the absorbent layer, wherein the adhesive layer bonds the waterproof layer and the absorbent layer together to form the protective barrier; and
at least one adhesive tab extending outward from an edge of the protective barrier, wherein the at least one adhesive tab removably attaches to a portion of protective barrier to hold the protective barrier around the arm of the patient during the blood pressure measurement.

2. The protective barrier of claim 1, wherein the waterproof layer is formed from one or more of a polyethylene material, a polypropylene material, a polyethylene terephthalate material, or a combination thereof.

3. The protective barrier of claim 1, wherein the absorbent layer is formed from one or more of a paper material, a natural fabric material, a synthetic fabric material, or a combination thereof.

4. The protective barrier of claim 1, wherein the protective barrier comprises a width between 4 to 12 inches.

5. The protective barrier of claim 1, wherein the protective barrier comprises a length selected between 8 to 16 inches.

6. The protective barrier of claim 1, wherein the protective barrier forms a tubular structure around the arm of the patient, and wherein the at least one adhesive tab holds the tubular structure around the arm of the patient.

7. A method of protecting an arm of a patient during a blood pressure measurement comprising:
wrapping a protective barrier around the arm of the patient prior to taking the blood pressure measurement;
overlapping a first edge with a second edge of the protective barrier to form a tubular structure around the arm of the patient; and
wrapping a blood pressure device around the arm of the patient, wherein the blood pressure device contacts an exterior surface of the protective barrier.

8. The method of claim 7, wherein the protective barrier comprises:
a waterproof layer defining an exterior waterproof surface of the protective barrier;
an absorbent layer defining an interior absorbent surface of the protective barrier; and
an adhesive layer disposed between the waterproof layer and the absorbent layer, wherein the adhesive layer bonds the waterproof layer and the absorbent layer together to form the protective barrier.

9. The method of claim 8, wherein wrapping the blood pressure device around the arm of the patient comprises positioning the blood pressure device in direct contact with the exterior waterproof surface of the protective barrier.

10. The method of claim 8, wherein wrapping the protective barrier around the arm of the patient comprises positioning the interior absorbent surface in direct contact with the arm of the patient.

11. The method of claim 8, wherein the waterproof layer is formed from one or more of a polyethylene material, a polypropylene material, a polyethylene terephthalate material, or a combination thereof.

12. The method of claim 8, wherein the absorbent layer is formed from one or more of a paper material, a natural fabric material, a synthetic fabric material, or a combination thereof.

13. The method of claim 7, wherein the protective barrier comprises a width between 4 to 12 inches, and wherein wrapping the blood pressure device around the arm of the patient comprises aligning the blood pressure device with the protective barrier such that at least 1 inch of the protective barrier extends outwardly from the blood pressure device.

14. The method of claim 7, wherein the protective barrier comprises a length selected between 8 to 16 inches, and wherein wrapping the protective barrier around the arm of the patient comprises wrapping the protective barrier around a circumference of the arm of the patient.

15. The method of claim 7, wherein overlapping the first edge with the second edge of the protective barrier comprises attaching at least one adhesive tab to the first edge and the second edge to hold the protective barrier around the arm of the patient.

16. A protective barrier sheet including a plurality of protective barriers for protecting an arm of a patient during a blood pressure measurement, the protective barrier sheet comprising:
a first protective barrier of the plurality of protective barriers;
a second protective barrier of the plurality of protective barriers adjacent to the first protective barrier; and
a perforation formed between the first protective barrier and the second protective barrier such that the first protective barrier is separated from the second protective barrier by tearing the protective barrier sheet along the perforation, each protective barrier of the first protective barrier and the second protective barrier comprise:
a waterproof layer defining an exterior waterproof surface of the protective barrier,
an absorbent layer defining an interior absorbent surface of the protective barrier,
an adhesive layer disposed between the waterproof layer and the absorbent layer, wherein the adhesive layer bonds the waterproof layer and the absorbent layer together to form the protective barrier, and
at least one adhesive tab extending outward from an edge of the protective barrier, wherein the at least one adhesive tab removably attaches to a portion of protective barrier to hold the protective barrier around the arm of the patient during the blood pressure measurement.

17. The protective barrier sheet of claim 16, wherein the waterproof layer is formed from one or more of a polyethylene material, a polypropylene material, a polyethylene terephthalate material, or a combination thereof.

18. The protective barrier sheet of claim 16, wherein the absorbent layer is formed from one or more of a paper material, a natural fabric material, a synthetic fabric material, or a combination thereof.

19. The protective barrier sheet of claim 16, wherein each protective barrier of the first protective barrier and the second protective barrier comprises a width between 4 to 12 inches.

20. The protective barrier sheet of claim 16, wherein the each protective barrier of the first protective barrier and the second protective barrier comprises a length selected between 8 to 16 inches.
